(19) United States
(12) Patent Application Publication
(10) Pub. No.: US 2019/0192737 A1
(43) Pub. Date: Jun. 27, 2019

(54) Paracrine-Enriched Collagen Fleece

(71) Applicant: SpinPlant, GMBH, Halle (Saale) (DE)

(72) Inventor: Timothy Ganey, Tampa, FL (US)

(21) Appl. No.: 15/852,207

(22) Filed: Dec. 22, 2017

(51) Int. Cl. A61L 27/44; A61L 27/54; A61L 27/24; A61L 27/58; A61L 27/12; A61L 27/36; A61L 27/38

(52) U.S. Cl. CPC A61L 27/44; A61L 2300/412; A61L 27/24; A61L 27/58; A61L 27/12; A61L 27/3654; A61L 27/365; A61L 27/3608; A61L 27/3612; A61L 27/3847; A61L 27/3852; A61L 27/3817; A61L 27/3821; A61L 27/3687; A61L 27/3691; A61L 2400/06; A61L 2300/41; A61L 2300/604; A61L 2430/02; A61L 2430/06; A61L 27/54

(57) ABSTRACT

A biological composition made from a mixture of biologic material intermixed with an electrospun matrix for direct implantation has a mixture of biologic material and a volume of an electrospun matrix of collagen. The mixture of biologic material has non-whole cellular components including vesicular components and active and inactive components of biological activity, cell fragments, cellular excretions, cellular derivatives, and extracellular components, or whole cells or combinations of the non-whole cellular components and whole cells. The mixture is compatible with biologic function. The volume of electrospun matrix of collagen is intermixed with the mixture of biologic material. The electrospun matrix forms a three-dimensional electrospun scaffold externally enveloping each of the non-whole cellular components, if any, and each of the whole cells, if any, of the mixture of biologic material to form a biological composition of paracrine-enriched collagen fleece.

### FIELD OF INVENTION

The present disclosure is directed to a biological composition generally, and to the preparation of enriched collagen matrices, notably those of electrospun collagen and purposefully used to deliver non-whole fractions of chondrocytes or cartilage matrices into a three-dimensional scaffold in the form of a fleece, but not by limitation also to methods for producing medical treatments such as tissue allograft procedures using the biological composition.

### BACKGROUND OF INVENTION

Cartilage exists in the human body initially as anlagen for bone formation. In well-defined anatomical and morphologic text, vascular invasion, bone matrix deposition, and sustained advance of bone tissue to the margins of the bone leaves an articulating surface. This surface is unique in its lack of vascular supply, lymphatic drainage, or innervation. Despite being richly cellularized with chondrocytes surrounded by profuse matrix, healing does not occur after the fetal period. As such, widespread attention is placed on repairing cartilage at the end of long bones.

Osteoarthritis is pervasive in the general population and attains a prevalence nearing 50% in adults 65 years old or older. Osteoarthritis is actually more involved than merely a disease of cartilage. FIG. 1 demonstrates the inclusion of not only the cartilage, but also the underlying subchondral bone, and the joint capsule which sustains the synovial fluid for lubricating the joint movement, and blocks vascular dialysate from entering the joint capsule. It is beyond the scope of discussion to fully elucidate the full mechanism as ongoing work shows that understanding while improving still has not demonstrated a single treatment that reverses the ravages of injury, inflammation, and destructive change that eventually completely erodes and ultimately dissolves the articular cartilage from the ends of bone.

Put forward in this invention is a method for repairing focal cartilage defects, replacing areas of degraded surface, and integrating an effective mechanism for staving additional osteoarthritic change by first filling the surface defect, and second offering a scaffold for stem cells from the bone to penetrate, integrate, and restore surface conditions.

### SUMMARY OF THE INVENTION

A biological composition made from a mixture of biologic material intermixed with an electrospun matrix for direct implantation has a mixture of biologic material and a volume of an electrospun matrix of collagen. The mixture of biologic material has non-whole cellular components including vesicular components and active and inactive components of biological activity, cell fragments, cellular excretions, cellular derivatives, and extracellular components, or whole cells or combinations of the non-whole cellular components and whole cells. The mixture is compatible with biologic function. The volume of electrospun matrix of collagen is intermixed with the mixture of biologic material. The electrospun matrix forms a three-dimensional electrospun scaffold externally enveloping each of the non-whole cellular components, if any, and each of the whole cells, if any, of the mixture of biologic material to form a biological composition of paracrine-enriched collagen fleece.

The electropsun scaffold deters attachment to other cells for a predetermined time, buffers inflammation, and retards or reduces premature differentiation of the whole cells of the mixture. The electrospun scaffold sustains regenerative potential and biologic function of the mixture during preservation and implantation and is configured to be metabolized after implantation after a predetermined time, the predetermined time is three or more days, preferably up to six days, but dependent on individual metabolic variations from patient to patient, time is to functional filling of a defect with cartilaginous material.

The mixture is mechanically selected or enhanced allogeneic biology material derived from articular cartilage or autologous cartilage material from which cells have been harvested and whose chondrocytes have been expanded in culture. The mixture of mechanically selected material derived from chondrocytes or articular cartilage further includes a select number of non-whole cell fractions including one or more of exosomes, transcriptomes, proteasomes, membrane rafts, lipid rafts. The combination of non-whole cell components with a select number of the non-whole cell fractions sustains pluripotency in both graft or host cells or combinations thereof, and the select number of the non-whole cell fractions sustains pluripotency in graft or host cells or combinations thereof includes differentiated committed cells and non-differentiated and non-committed cells. The biological composition is predisposed to demonstrate or support elaboration of active volume or spatial geometry consistent in morphology with that of articular cartilage, and further extends regenerative resonance that complements or mimics tissue complexity.

In one embodiment, the mixture is treated in a protectant prior to preservation or cryopreservation or freeze drying. The protectant creates a physical or electrical or chemical gradient or combination thereof for tissue regeneration, wherein the gradient has a physical characteristic of modulus or topography such as charge density, field shape or cryo- or chemo-taxic tendencies, or wherein the gradient has a chemical characteristic of spatially changing compositions of density or species of functional molecules, wherein the molecules can offer a fixed catalytic function as a co-factor or wherein the gradient has an electrical characteristic of charge based or pH based or electron affinities that confer metastability in biologic potential. The articular cartilage derived from a cadaver has separation-enhanced non-whole cell fractions vitality including one or more of the following: separating the fractions from cells heightens their vitality, reversing “arrest” of donors, accentuating responsive molecular coupling, matrix guarding in neutralizing inflammation or providing a basis for metabolic satience by balancing stimulus for repair. The regenerative resonance occurs in the presence or absence of a refractory response. The mixture creates a physical or electrical or chemical gradient or combination thereof for tissue regeneration, the gradient has a physical characteristic such as modulus or topography, the gradient has a chemical characteristic such as spatially changing compositions of density or species of functional molecules, or the gradient has an electrical characteristic such as charge based or pH based. The vesicular components can be organelle fragments. The active and inactive components of biological activity can be extants of the human metabolome. The composition can be maintained at ambient temperature prior to freeze drying.

The mixture of biologic material intermixed with the electrospun matrix scaffold forms the three-dimensional scaffold for direct implantation wherein said scaffold that has dimensions qualifying between non-zero porosity and non-zero structure. The electrospun collagen further has hydroxyapatite.

The present invention allows for a method of making a biological composition comprises the steps of: collecting, recovering and processing articular cartilage from a cadaver donor or an autologous sample; mechanically separating cellular and non-cellular components from the cartilage; concentrating by centrifugation and filtering; separating by density gradient centrifugation; collecting non-cellular fractions or non-cellular components or combinations thereof of predetermined density; washing the non-cellular fractions or non-cellular components or combinations thereof to create a chondrocyte mixture; quantifying non-whole cell fraction concentration exceeding zero; suspending to a predetermined concentration in a polyampholyte cryoprotectant; applying the suspended chondrocyte mixture to an appropriately dense electrospun collagen material; freeze-drying the mixture at a predetermined controlled rate; and aseptically packaging the resultant material.

The method of preparing the mixture for use made according to the present invention includes the steps of: implanting the diluted mixture with or without the chondrocyte mixture being intermixed by packing, injection or any other suitable means into a patient.

### DEFINITIONS

DNase—deoxyribonuclease is any enzyme that catalyzes the hydrolytic cleavage of phosphodiester linkages in the DNA backbone, thus degrading DNA.

DMEM, DMEM/LG—Dulbecco's Modified Eagle Medium, low glucose. Sterile, with: Low Glucose (1 g/L), Sodium Pyruvate; without: L-glutamine, HEPES (4-(2-hydroxyethyl)-1-piperazineethanesulfonic acid)

Dimethyl sulfoxide (DMSO) is an organosulfur compound with the formula (CH3)2SO. This colorless liquid is an important polar aprotic solvent that dissolves both polar and nonpolar compounds and is miscible in a wide range of organic solvents as well as water. It has a relatively high melting point.

DPBS—Dulbecco's Phosphate Buffered Saline.

CBT-MIXER—Mixing blade for Cancellous Bone Tumbler Jar.

Chimera—A genetic chimerism or chimera (also spelled chimaera) is a single organism composed of cells with distinct genotypes.

Cold Media—Media used during the preparation of vertebral bodies for initial processing.

Cryopreserved—Tissue frozen with the addition of, or in a solution containing, a cryoprotectant agent such as glycerol, or dimethylsulfoxide, or carboxylated poly-1-lysine.

Freeze Dried/Lyophilized—Tissue dehydrated for storage by conversion of the water content of frozen tissue to a gaseous state under vacuum that extracts moisture.

Normal Saline—0.9% Sodium Chloride Solution.

Packing Media—Media used during initial processing and storage of the processed vertebral bodies prior to bone decellularization.

Paracrine—of, relating to, promoted by, or being a substance secreted by a cell and acting on adjacent cells.

PBS—Phosphate Buffered Saline.

Processing Media—Media used during bone decellularization that may contain DMEM/Low Glucose no phenol red, Human Serum Albumin, Heparin, Gentamicin and DNAse.

### BRIEF DESCRIPTION OF THE DRAWINGS

The patent or application file contains at least one drawing/photograph executed in color. Copies of this patent or patent application publication with color drawing(s) will be provided by the Office upon request and payment of the necessary fee.

Certain embodiments of the present technology are illustrated by the accompanying figures. It will be understood that the figures are not necessarily to scale and that details not necessary for an understanding of the technology or that render other details difficult to perceive may be omitted. It will be understood that the technology is not necessarily limited to the particular embodiments illustrated herein.

FIG. 1 is a schematic diagram of a system of the present disclosure and defines the scope of application continuous with an articulating joint with both bone, cartilage, and capsular structures.

FIG. 2A is a plan view of nano-spun fibrillar Type I collagen.

FIG. 2B is a plan view of nano-spun fibrillar Type I collagen with hydroxyapatite.

FIG. 3 is an image of cell migration into the three-dimensional electron spun scaffold of porous collagen.

FIG. 4A is an image of cell matrix adhesion.

FIG. 4B is an image showing multiple adhesion contacts of the cell matrix.

FIG. 5A is a photograph of the composition after 8 weeks storage.

FIG. 5B is an enlarged photograph of the composition taken from FIG. 5A.

FIG. 6 is an x-ray showing a defined deficit repair on a join.

FIG. 7A is a photograph of one example of a cartilage implantation with autologous chondrocyte transplantation.

FIG. 7B is a second example of mosaic-plasty.

FIG. 8 is a schematic view of cell supernatant source of exosome.

FIG. 9 is a schematic view of the electrospun collagen as a scaffold for the biologic mixture having acellular exosomes.

FIG. 10 is a graph showing the effect of acoustic shock waves on exosome activity.

FIG. 11 is a diagrammatic view of the lipid head shift with different pH changes.

FIG. 12 is an illustration of forming repair disks of the composition taken from a sheet of the composition.

FIG. 13 illustrates the use of stacking the disks to match the repair depth.

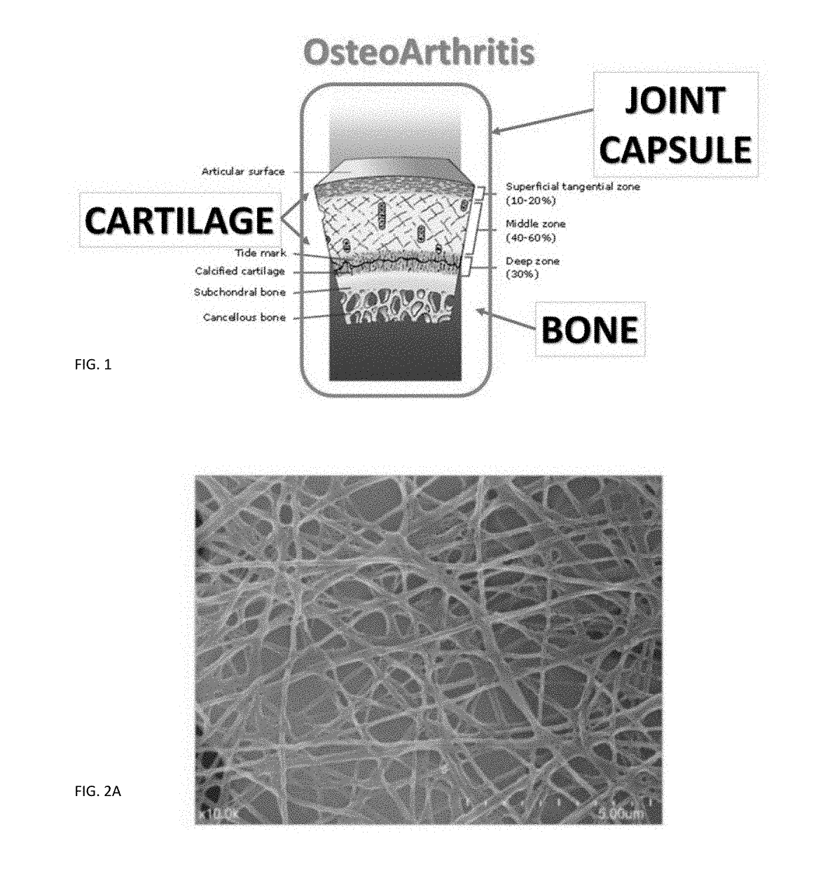

### DETAILED DESCRIPTION OF THE INVENTION

Generally speaking, the present disclosure is directed to methods for producing fibrillated tissue matrices. Example methods comprise any combination or permutation of cell culture, shock wave stimulation, pulsed electric field stimulation, liquid supernatant collection, matrix recombination with nano-fibrillar collagen and liquid supernatant, and lypoholization—just to name a few. The resultant electrospun collagen fleece will be exosome rich and cytokine rich, and can be used in medical procedures such as cartilage allograft procedure.

The invention relies on a scaffold that is produced from electrospun collagen that can be made of varying thicknesses, falling within ranges of non-zero dimension, and extending to include dimensions between 500-1000 micrometers, 1 and 5 millimeters, and in some applications to be offered in ranges of 5 to 10 mm in total thickness. The nano-spun has been well characterized and consists of individual fibers varying in ranges of non-zero dimension to less than 1 micrometer. In some instances, the dimensions of the fibers might fall between 1000 and 2000 nanometers (FIGS. 2A, 2B).

In some embodiments, electrospun collagen matrix is created using a shockwave instrument to stimulate chondrocyte cells in culture. Chondrocyte segments, including whole and non-whole cell components are retained in a vessel apparatus and the vessel apparatus is at least partially filled with a carrier fluid. The vessel apparatus is configured in some embodiments to absorb and transmit the physical stimulus through the carrier fluid containing the chondrocytes while maintaining a temperature of the contents approximately within a safe zone temperature range that reduces a likelihood of deleterious damage to component viability such as exosomes, cytokines, growth factors, sulfated proteoglycans, and other similar tissue components that might result from slight and transient increases of temperature when a corresponding increase in pressure is achieved with the shockwave instrument. In this regard, other aspects of the vessel might integrate thermocouple modulation and temperature control. Additional assets to the concept would be an availability to modulate the partial pressure of gasses, including nitrogen, carbon dioxide, oxygen, nitrous oxide, and others used to modulate and optimize cell culture conditions.

In one or more embodiments, chondrocytes and suitable carrier such as saline, or cell culture media are combined into a vessel that can be safely contained.

This invention entails the manufacturing of an enriched cell culture milieu derived from human chondrocytes that are imbued with biologic potential gained from specified mechanical, electrical, and magnetic transfers of energy to the material defined by optimized processing. Chondrocytes can be obtained from autologous donors of tissue cartilage explants, or from younger, allogeneic donors. For allogeneic tissue sourcing, full body donors with no joint replacements are preferred. Donor medical and social history are screened for medical conditions which may hinder the intended purpose of the final product with contaminants and those are excluded.

Accentuation of chondrocyte specific properties including acellular packaging components, vascular sparing, matrix elaboration, directed morphogenesis and lateral transfer of genetic information can occur as a result of non-invasive treatment of cartilage tissue in general and chondrocyte tissues in particular before downstream processing.

Treatment for accentuation of bioenergetics may be mechanical, atmospheric or adjustment to membrane charge. Examples of tissue response to pressure waves, electric fields, magnetic fields, pressure variations, and ion streaming induction with pH are known in the literature and in this application, are attuned to energy transfer options that result in liposomal exchange, clathrin-based exosome expulsion, and gene tuning to specific protein translation. Some methods which promote these effects are PEMF, shockwave, negative pressure, tuned chirality, pH-induced shock and membrane coating.

The cell culture process of cartilage tissue exposes proteins and other intrinsic growth factors that precipitate signals that promote matrix expression and expand cells in specific chondrocyte lineages. One of the goals of the present invention is to support long term integration of therapeutic effect in regenerative applications that will offer clinical solutions for cartilage defects at articulating surfaces between long bones. Moreover, the mechanisms envisioned to extend optimized conditions of the bioenergized material will sustain and subsidize sufficient stimuli to provide metabolic support and integrated grafting of host and donor tissues.

The process of enhancement should precede the processing as the goal is to stimulate the cells to expose exosomes, and attune genetic machinery to building cartilage. The intent is to encourage microRNA, and exosomal packages that are chondrocyte inductive, as well as for the machinery to translate proteins until the process is stopped for harvest and production.

Concepts and contexts envisioned in material optimization use scaffold allograft material as a sump for biologically viable components that are energetically stimulated in situ while viability remains. Tissue processing achieves sufficient differences in matrix exosomes, in cell membrane and DNA packaging, and in the contents of the allograft, which at the most principal level is a biologic reservoir of tissue specific chemical matrices.

All manufacturing, including recovery and further processing of the cultures is performed using aseptic technique. Samples are taken for microbiological cultures immediately after the excision of tissue to be used for processing of the components. All manufacturing prior to and during the packaging process is performed inside a monitored ISO Class 5 suite.

Various processes are used to stimulate the bone that have been shown in vitro to demonstrate a biological response. In particular, previous work using PEF with defined frequency distribution of 1.22 mV/cm2 can be achieved by calculating the resistance of the solutions surrounding the biologic material and voltage adjusted accordingly. In previous work, conductivity of 83.5 ohm/cm was used to define volume, signal generation, and process time. No additional materials other than a wet holding solution are used in the process.

A frequency generator is used to energize coils to create a “pulsed” electromagnetic field. The field is imposed across the containers diameter or length.

Exposing the container to static, strong magnetic fields in a single direction across the diameter or length of the container.

A compressional wave of high amplitude is applied to the container. The shock wave will propagate through the medium; causing an abrupt, nearly discontinuous change in pressure, temperature and density of the medium.

The use of shockwave causes changes in pressure of the medium to which it is applied. This may also be applied as its own treatment by creating a vacuum within the container using conventional technology.

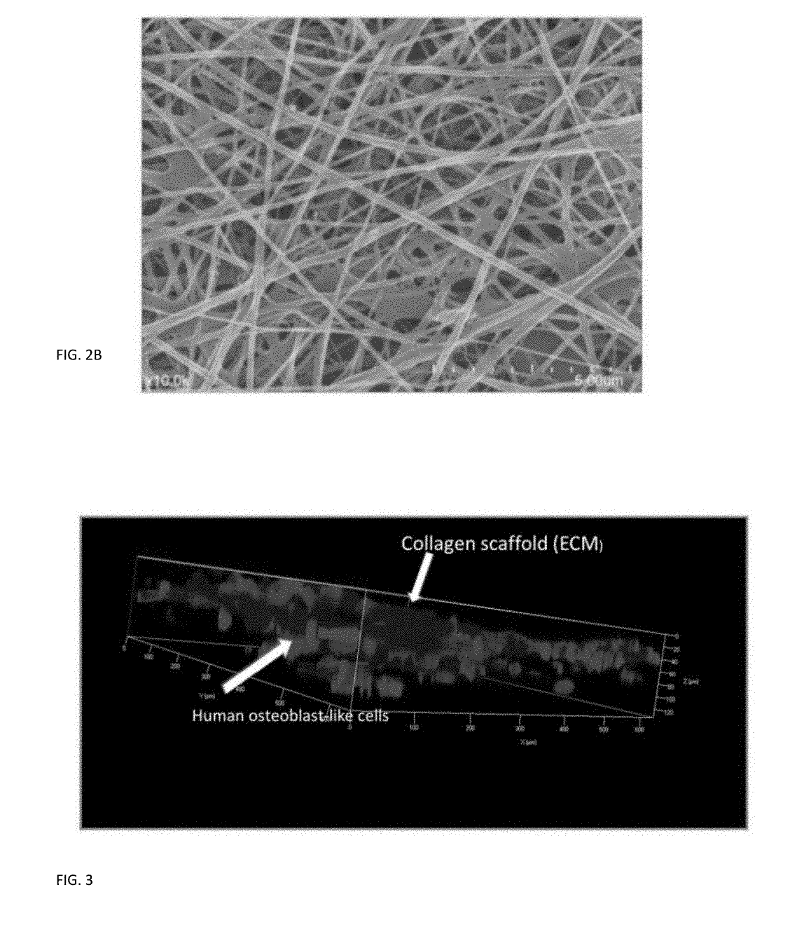

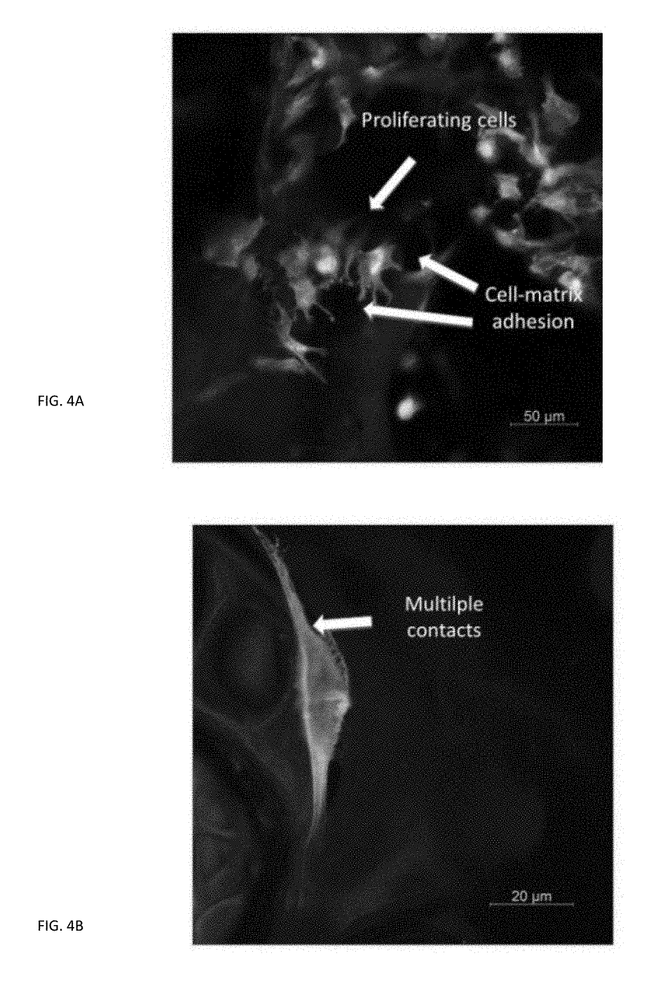

Previous work in the laboratory has shown that cells are capable of integrating the fiber and enriching in dimension not only surface but interior of the matrices as well (FIG. 3). Magnification of the cell attachments demonstrate a matrix affinity that supports cell proliferation (FIGS. 4A, 4B).

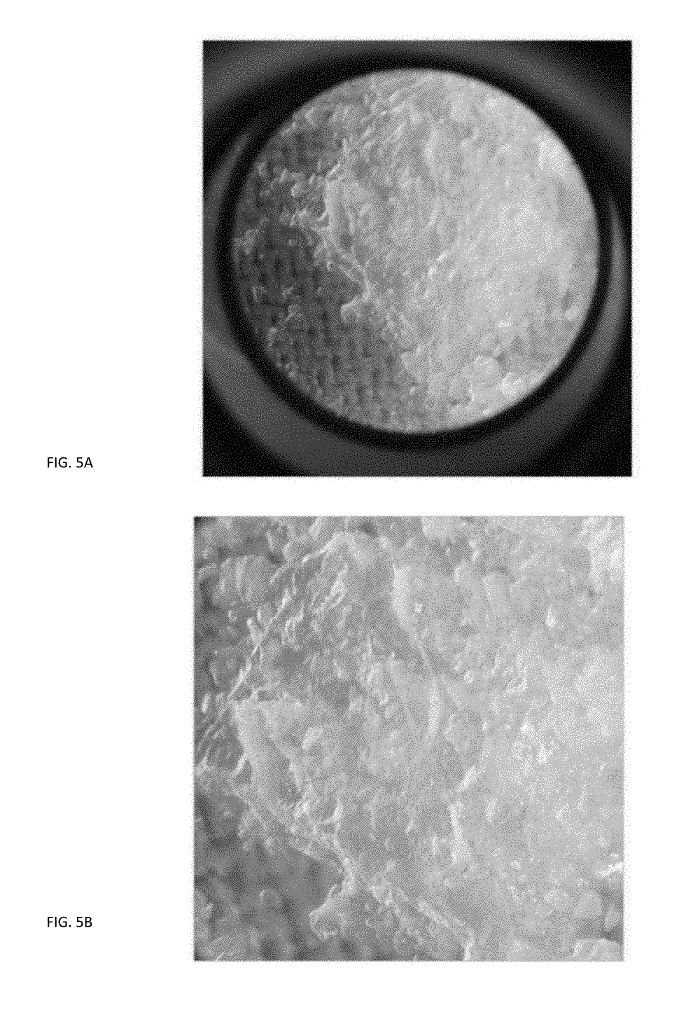

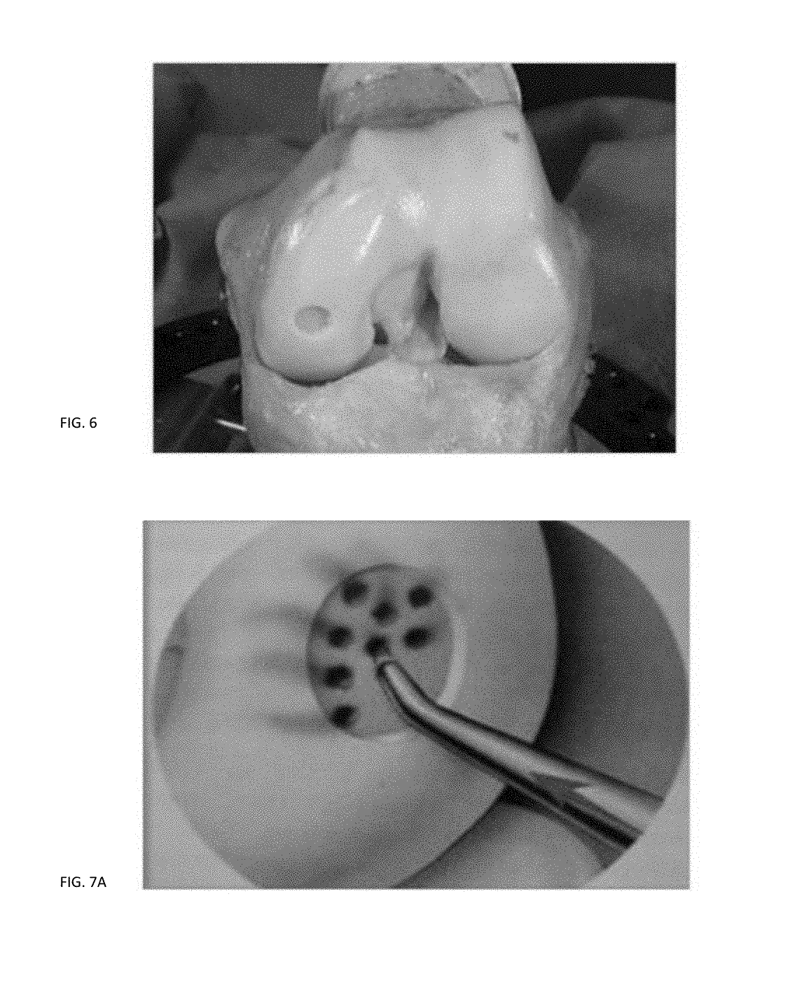

Electrospun collagen, when crosslinked by various means, has shown surprising durability when submitted to conditions comparable to body placement. Mixed with demineralized bone matrix (DBM), it is clear that the collagen supports its structure without melting, shrinking, or in any other way adopting a morphology separate from wettable collagen (FIGS. 5A, 5B). This material has been mixed with cells, and in that combination, has been shown to created adaptable scaffolds that are sufficient to prevent void occlusion with fibrous scar tissue.

Strategies for cartilage repair have been limited by the reticence of the tissue to undergo regenerative process, and reconstitute both a cellular and a matrix-cell mixture that is load bearing and seamless with extant tissue. The advent of arthroscopic techniques for surgery, the ability to do surface repairs and accommodate the removal of loose cartilage, hypertrophic synovium, and fissured tissue has hastened the number of surgeries, made some incremental process in reducing pain, but has not provided sufficient protection to avoid further degradation and joint destruction. With those degenerating changes, the synovial lining is inflamed, the nerve sensation at the joint margins are exacerbated, and patients have unremitting pain.

The insight into this invention comes from the lack of available scaffold, the inert nature of substitutes, and is derived in part by intent to prevent further degradation by techniques where healthy tissue is harvested and imposed into areas of damage.

Two technologies, one of which is referred to above, have been used exhaustively (FIGS. 7A, 7B). Autologous chondrocyte implantation/transplantation (ACI/ACT) was developed within a context of using patients own cells by biopsy, expanding them in culture, and then placing them into a well prepared, geometrically idealized realm for their implantation. This technology draws on the shortcomings of time to expand, technologically demanding and costly business modeling, and limitations of outcome to concise, well demarcated articular cartilage wounds.

An alternative technique was developed by Lazlo Hangody that has been referred to as mosaicplasty. The concept is straightforward and suggests that non-loaded surfaces of cartilage can be harvested and then placed into areas that have been prepared. By using serial die cutting tools, the filling in near complete and the resultant surface becomes inlaid as one might consider a mosaic (FIG. 7B). Combined with micro-picking of the subchondral bone, the goal of the surgeon is to stimulate healing from the marrow cavity, and use the stem cells incumbent in marrow to provide the cell source into the filled defect. While some limited successes have been achieved with both methods, the solution is imperfect. ACI.ACT is time delayed and costly, and despite a good fit in some cases with the mosaic placement, the articular surface from which the donor tissue was harvested continues to exude plasma proteins into the joint that affects the capsule as well as the underlying subchondral bone.

This invention proposes a solution that is cost effective, can be both autologous or allogeneic, and utilizes assets of the cell expansion technology to extend paracrine factors, and captures these factor with high affinity in the scaffold formed from electrospun collagen. The scaffold appropriates the geometric sparsity of cartilage regarding defined structure, and with 90% porosity, room for matrix deposition with the tensile constrictions of the nano-fibers offers a swelling pressure in addition to a shape adapting enclosure to repair articular cartilage defects.

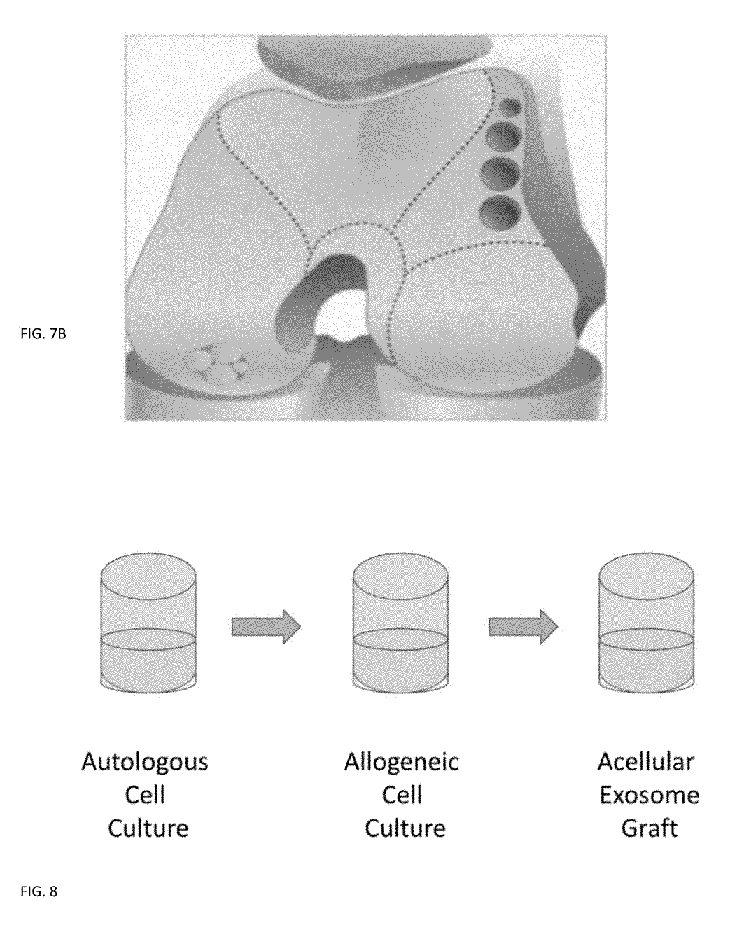

The process is supernatant based, easily allowing separation of cells. Within the context of this discussion, means of extending content, or increasing the shedding of exosome, paracrine, membrane, and proteasome components have been identified. In a short illustration, the context of culture, stimulation, and developing an acellular graft are highlighted (FIG. 8).

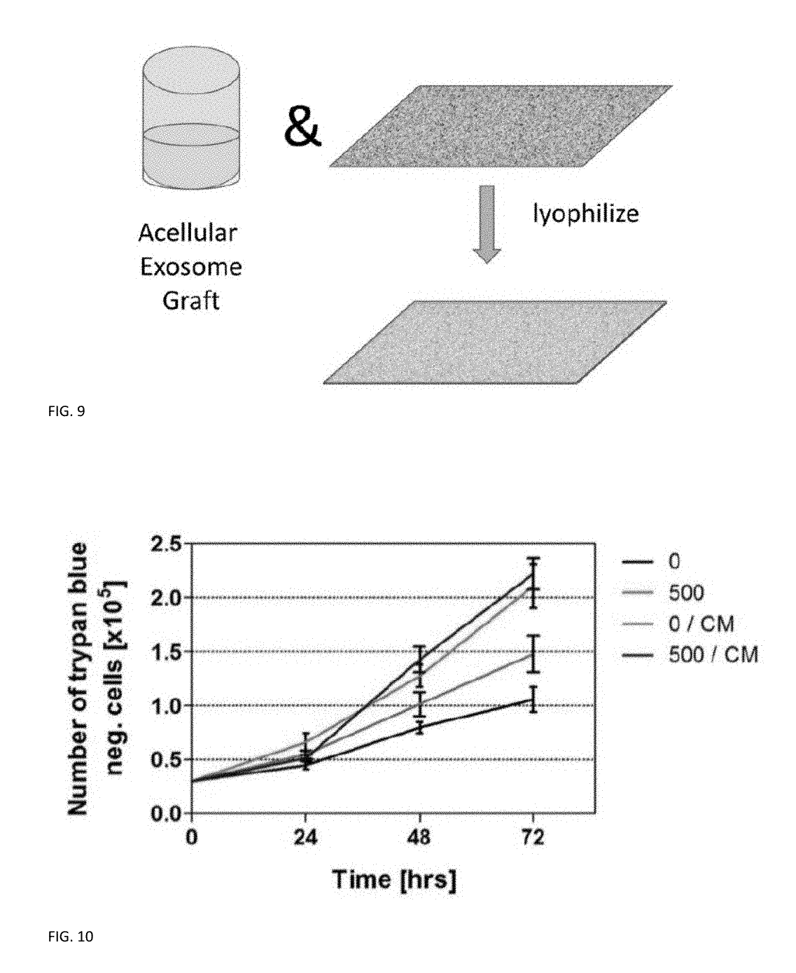

The liquid supernatant is rich in paracrine factors, and size limitations of exosomes with the realm of 40-100 nanometers offer differential centrifugation options to removing whole cell debris without eliminating the solute, or suspensions of the matrix. By attending the supernatant in a series of steps of 100,000 G centrifugation, washing, and resuspension, it is possible to concentrate paracrine cell factors, and then saturate electrospun collagen fleeces with the pure chondrocyte cell factors as noted earlier (FIG. 9).

Magnetic fields, shockwaves or pressure enhance the shedding of exosomes and microvesicles. These acellular components are known to represent the cell phenotype and signaling from which they arose. Increasing the concentration of these subcellular and acellular components as more readily available signaling adjuncts enriches the particulate in determining phenotypic complexity and regenerative capacity.

Circulating currents in the body have differential biological effects on healing and stimulation based on the induced current density. The use of 1 to 1000 mA/m² PEMF promotes exosome and microvesicle shedding in cells, enhancing the deliverable signals within the cells to neutralize inflammatory bias and normalize tissue identity. This enhancement is maintained during the processing of the cells in culture and adapted to stimulation adopted and carried forth on the scaffold.

The use of shockwaves has been shown to condition the media in which the cells are suspended with microvesicles and exosomes. Media conditioned with shockwave treated cells positively affects the cells count and viability of target cells as shown in FIG. 10.

Additionally, the increased concentration of exosomes, microvesicles and available signaling molecules shed from the particulate contains specific chondro-inductive signals inherent in the donor bone before processing.

Exosomes, microvesicles and other components shed during treatment contain representative DNA, RNA and proteins of the source cell. Increased concentration of these components promotes lateral signaling, including but not limited to: access by endocytosis or passive entry to recipient cells, extracellular and intracellular receptor activation or inactivation through multiple mechanisms. Such mechanisms may include translation of provided genetic material, modification of resident proteins by provided enzymes or binding factors, binding to receptors, cleavage of receptors, RNA binding for activation or inactivation and DNA binding for activation or inactivation. All such activities represent the promotional activities of the source cell. In the case of the treatments presented here, the source cells are those resident in chondrocyte cells, or from isolated articular cartilage from donor tissues.

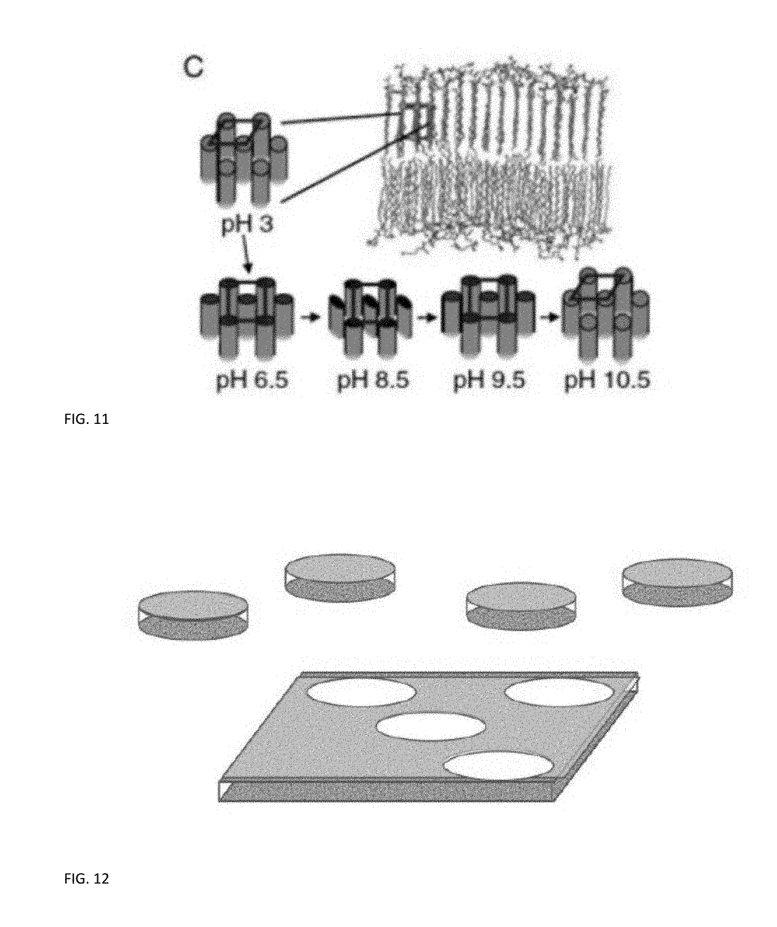

It is known that pH changes induce cellular stress. As a response to stress, cells shed exosomes, microvesicles and other components. Additionally, there have been studies showing that pH shock induces a stem cell response in the cells. Bilayers are made of two layers of amphiphile molecules which possess a charged hydrophilic head and a hydrophobic tail. The density and arrangement of the molecules within the membrane determine the membrane's porosity, strength, and other properties. Most molecules in the membrane do not respond to a change in acidity. For the molecules that are affected, the charge of the molecules' heads changes in that their two-dimensional crystallization morphs from a rectangular-patterned lattice (basic solutions) to a hexagonal lattice (acidic solutions). Membranes with a higher symmetry, such as hexagonal, are stronger and less brittle than those with lesser symmetry (FIG. 11).

The change in pH also alters the bilayers' thickness and the compactness of the molecules. Changing the density and spacing of molecules within membranes helps control the encapsulation and release efficiency of molecules inside a vesicle. Charge adjustment causes an immediate increase in perinuclear vesicles which are lipid rich, can be extracted and maintain their identity.

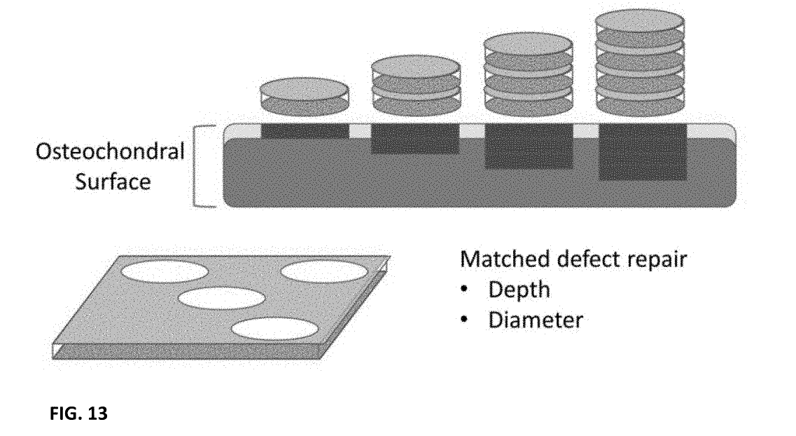

With that introduction to the variations of enhancement, the invention is demonstrated to sow the provision for creating sheets of collagen fleece, for prescribing exacting matrices in context of the dimension of the injury and in replenishing the surface with appropriate thickness and diameter (FIG. 13).

The invention overcomes the delays incumbent to cell expansion, incorporates advantages of direct template repair without taking healthy tissue, and further ascends value in the porosity of the material offering a wicking, absorbent, fluid and cell reservoir for sustaining the regenerative intention.

By way of non-limiting example, the enriched collagen scaffold can be used to create a cartilage allograft used to for grafting into a patient. In some embodiments, additional compounds can be introduced into the fibrillated matrix such as a tissue growth enhancing product, a medicament, a filler, or any combinations thereof.

While this technology is susceptible of embodiment in many different forms, there is shown in the drawings and has been described in detail several specific embodiments with the understanding that the present disclosure is to be considered as an exemplification of the principles of the technology and is not intended to limit the technology to the embodiments illustrated.

The electrospun matrix of collagen can preferably be made in accordance with the teachings in U.S. Pat. No. 9,522,507 B2 which is being incorporated herein by reference in its entirety.

In a further embodiment, the carrier material is solved or dispersed in at least one liquid chosen from the group of water, alcohols like methanol or ethanol, aqueous solutions of acids or bases like acetic acid or sodium hydroxide and organic solvents like acetone or 1,1,1,3,3,3-hexaflouoro-2-propanol to produce a carrier material solution.

The term “carrier material solution” also encompasses “carrier material dispersions”. This means, it is not necessary that the carrier material is ideally solved in an according liquid. If an essentially stable dispersion of the carrier material in an according liquid is established, electrospinning can also take place.

In a further embodiment, the carrier material is collagen, a mixture of collagen and hydroxy apatite, gelatin, alginates, chitosan, silk, cellulose, polyurethane, a polyester, polycaprolactone, polylactide, polypyrrole, polyaniline, polyacetylene, polythiophene, a copolymer of the preceding polymers and/or a copolymer bearing carboxylic acid groups and/or amine groups.

Well-suited collagens are collagen type I, II, III, V, or XI, wherein type I collagen is particularly well suited. The collagen might for example have a human, bovine, equine, ovine or fish origin or can be an artificial collagen resembling human, bovine, equine, ovine or fish collagen, or might consist of collagen, or collagen fibrils that have been expressed in culture by vector incorporation without limitation to mammalian source.

In a further embodiment, the carrier material solution further comprises at least one auxiliary substance. This auxiliary substance is chosen from the group consisting of osteoinductive substances, electrically conductive substances, electrically semiconductive substances, electrically insulating substances, antibacterial substances, antiviral substances, antifungal substances, ceramics (like, e.g., hydroxyapatite ceramics), barium, bromine, copper, niobium, lithium, germanium, titanium, lead, zirconium, silicon, silver, zinc, polyurethane, silver hydrogen sulfate, gallium orthophosphate (GaPO₄), langasite (La₃Ga₅SiO₁₄), barium titanate (BaTiO₃), lead titanate (PbTiO₃), lead zirconate titanate (Pb[ZrₓTi₁₋ₓ]O₃ 0<x<1), potassium niobate (KNbO₃), lithium niobate (LiNbO₃), lithium tantalate (LiTaO₃), sodium tungstate (Na₂WO₃), Ba₂NaNb₅O₅ and Pb₂KNb₅O₁₅.

In a further embodiment, the electrospun carrier material is washed after having been removed from the collector. By washing, remainders of non-reacted crosslinker or reaction co-products can be removed, thus improving the biocompatibility of the obtained product. Washing can be performed by an aqueous solution of an alcohol like for example ethanol in a concentration of for example 80%, 70%, 60% or 50% (v/v). Further suited washing solutions are low salt buffers like phosphate buffered saline (PBS). Washing can be performed in a two-step manner by first using an alcoholic solution and afterwards a low salt buffer (or first a low salt buffer and afterwards an alcoholic solution) as washing solutions.

In an alternative embodiment, no washing step is necessary due to the specific crosslinker chosen and the concentration in which it is used.

The object is further achieved by a sheet material having the features explained in the following. Such a sheet material has a three-dimensional surface structure for enabling tissue growth. It is in particular obtainable by a method according to the above explanations. The sheet material comprises voids and struts surrounding the voids, wherein the struts are made of a carrier material being built up from nanofibers having a diameter of less than 1200 nm. According to an aspect of the invention, the struts have a thickness in the range of 100 to 600 μm, wherein the average distance between the edges of two adjacent struts is in the range of 200 to 750 μm. In an embodiment, the nanofibers might have a diameter of less than 1100 nm, in particular less than 1000 nm, in particular less than 900 nm, in particular less than 800 nm, in particular less than 700 nm, in particular less than 600 nm, in particular less than 500 nm, in particular less than 400 nm, in particular less than 300 nm. The thickness of the struts might lie in an embodiment in the range of 130 to 550 μm, in particular of 200 to 500 μm, in particular of 230 to 450 μm, in particular of 300 to 400 μm. The average distance might in an embodiment be in the range of 300 to 600 μm, in particular of 400 to 500 μm.

In an embodiment, the ratio of material volume to strut volume is in the rage of 15 to 30, in particular of 17 to 25. Thus, there are many voids present in the overall material, leading to a high porosity of the material.

The nanofibers building up the sheet material might be essentially aligned to each other in one direction. Alternatively, the fibers can be randomly orientated, or stacked matters of fibers can bear respective orthogonal relationships with adjacent levels.

In an embodiment, the average cross-sectional area of a single void is in the range of 2000 to 100000 μm<2>. Such a range is suited for a cell to adhere to the material surrounding an according void. The average cross-sectional area of a single void might also be in the range of 5000 to 75000 μm<2>, in particular of 10000 to 60000 μm<2>, in particular of 20000 to 50000 μm<2>, in particular of 30000 to 40000 μm<2>.

In an embodiment, the distribution of the voids or pores is not too regular in order to produce a material which reflects the irregularity of natural tissue. For example, smaller voids might be located next to larger voids so that the material strength increases as compared to material in which distinct areas of large voids and distinct areas of small voids exist.

While various embodiments have been described above, it should be understood that they have been presented by way of example only, and not limitation. The descriptions are not intended to limit the scope of the technology to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the technology as defined by the appended claims and otherwise appreciated by one of ordinary skill in the art. The scope of the technology should, therefore, be determined not with reference to the above description, but instead should be determined with reference to the appended claims along with their full scope of equivalents.

What is claimed is:

1. A biological composition made from a mixture of biologic material intermixed with an electrospun matrix for direct implantation comprising:
a mixture of biologic material having non-whole cellular components including vesicular components and active and inactive components of biological activity, cell fragments, cellular excretions, cellular derivatives, and extracellular components, or whole cells or combinations of the non-whole cellular components and whole cells, wherein the mixture is compatible with biologic function;
a volume of an electrospun matrix of collagen intermixed with the mixture of biologic material; and
wherein the electrospun matrix forms a three-dimensional electrospun scaffold externally enveloping each of the non-whole cellular components, if any, and each of the whole cells, if any, of the mixture of biologic material to form a biological composition of paracrine-enriched collagen fleece.

2. The biological composition of claim 1 wherein the electropsun scaffold deters attachment to other cells for a predetermine time.

3. The biological composition of claim 1 wherein the electrospun scaffold buffers inflammation.

4. The biological composition of claim 1 wherein the electrospun scaffold retards or reduces premature differentiation of the whole cells of the mixture.

5. The biological composition of claim 1 wherein the electrospun scaffold sustains regenerative potential and biologic function of the mixture during preservation and implantation.

6. The biological composition of claim 1 wherein the electrospun scaffold is configured to be metabolized after implantation after a predetermined time.

7. The biological composition of claim 6 wherein the predetermined time is three or more days.

8. The biological composition of claim 7 wherein the predetermined time is up to six days.

9. The biological composition of claim 1 wherein the mixture is mechanically selected or enhanced allogeneic biology material derived from articular cartilage or chondrocyte culture or autologous cartilage material from which cells have been harvested and whose chondrocytes have been expanded in culture.

10. The biological composition of claim 9 wherein the mixture of mechanically selected material derived from chondrocytes or articular cartilage further includes a select number of non-whole cell fractions including one or more of exosomes, transcriptomes, proteasomes, membrane rafts, lipid rafts.

11. The biological composition of claim 10 wherein the combination of non-whole cell components with a select number of the non-whole cell fractions sustains pluripotency in both graft or host cells or combinations thereof.

12. The biological composition of claim 11 wherein the select number of the non-whole cell fractions sustains pluripotency in graft or host cells or combinations thereof includes differentiated committed cells and non-differentiated and non-committed cells.

13. The biological composition of claim 12 wherein the biological composition is predisposed to demonstrate or support elaboration of active volume or spatial geometry consistent in morphology with that of articular cartilage.

14. The biological composition of claim 1 wherein the biological composition extends regenerative resonance that compliments or mimics tissue complexity.

15. The biological composition of claim 1 wherein the mixture is treated in a protectant prior to preservation or cryopreservation or freeze drying.

16. The biological composition of claim 15 wherein the protectant creates a physical or electrical or chemical gradient or combination thereof for tissue regeneration.

17. The biological composition of claim 16 wherein the gradient has a physical characteristic of modulus or topography such as charge density, field shape or cryo- or chemo-taxic tendencies.

18. The biological composition of claim 17 wherein the gradient has a chemical characteristic of spatially changing compositions of density or species of functional molecules, wherein the molecules can offer a fixed catalytic function as a co-factor.

19. The biological composition of claim 18 wherein the gradient has an electrical characteristic of charge based or pH based or electron affinities that confer metastability in biologic potential.

20. The biological composition of claim 9 wherein the articular cartilage which is derived from a cadaver has separation-enhanced non-whole cell fractions vitality including one or more of the following: separating the fractions from cells heightens their vitality, reversing “arrest” of donors, accentuating responsive molecular coupling, matrix guarding in neutralizing inflammation or providing a basis for metabolic satience by balancing stimulus for repair.

21. The biological composition of claim 20 wherein the regenerative resonance occurs in the presence or absence of a refractory response.

22. The biological composition of claim 1 wherein the mixture creates a physical or electrical or chemical gradient or combination thereof for tissue regeneration.

23. The biological composition of claim 32 wherein the gradient has a physical characteristic such as modulus or topography.

24. The biological composition of claim 23 wherein the gradient has a chemical characteristic such as spatially changing compositions of density or species of functional molecules.

25. The biological composition of claim 24 wherein the gradient has an electrical characteristic such as charge based or pH based.

26. The biological composition of claim 1 wherein the vesicular components can be organelle fragments.

27. The biological composition of claim 1 wherein active and inactive components of biological activity can be extants of the human metabolome.

28. The biological composition of claim 27 wherein the composition is maintained at ambient temperature prior to freeze drying.

29. The biological composition of claim 1 wherein the mixture of biologic material intermixed with the electrospun matrix scaffold forms the three-dimensional scaffold for direct implantation wherein said scaffold that has dimensions qualifying between non-zero porosity and non-zero structure.

30. The biological composition of claim 1 wherein the electrospun collagen further has hydroxyapatite.

31. A method of making a biological composition comprises the steps of:
collecting, recovering and processing articular cartilage from a cadaver donor or an autologous sample;
mechanically separating cellular and non-cellular components from the cartilage;
concentrating by centrifugation and filtering;
separating by density gradient centrifugation;
collecting non-cellular fractions or non-cellular components or combinations thereof of predetermined density;
washing the non-cellular fractions or non-cellular components or combinations thereof to create a chondrocyte mixture; quantifying non-whole cell fraction concentration exceeds zero; suspending to a predetermined concentration in a polyampholyte cryoprotectant;
applying the suspended chondrocyte mixture to an appropriately dense electrospun collagen material; freeze-drying the mixture at a predetermined controlled rate; and aseptically packaging the resultant material.

32. The method of preparing the mixture for use made according to the method of claim 31 by the steps of:
implanting the diluted mixture with or without the chondrocyte mixture being intermixed by packing, injection or any other suitable means into a patient.
